(19) United States
(12) Patent Application Publication
(10) Pub. No.: US 2013/0338586 A1
(43) Pub. Date: Dec. 19, 2013

(54) INJECTION DEVICE

(71) Applicants: Lisa Moreno Dickinson, Westchester, PA (US); Michael Anthony Quick, Marlton, NJ (US)

(72) Inventors: Lisa Moreno Dickinson, Westchester, PA (US); Michael Anthony Quick, Marlton, NJ (US)

(21) Appl. No.: 13/912,931

(22) Filed: Jun. 7, 2013

Related U.S. Application Data
(60) Provisional application No. 61/656,702, filed on Jun. 7, 2012.

(51) Int. Cl. A61M 5/42

(52) U.S. Cl. CPC A61M 5/422
USPC 604/112; 604/218

(57) ABSTRACT

A injection device may be disclosed. The device may include an outer casing, a needle, and a plurality of projections surrounding the needle. The needle may be adjustable between a first position and a second position. A vibration motor may be disposed within the device to impart vibration to the device. Decorative indicia may be provided on the exterior of the device. In use, the injection device may provide sensory stimuli to distract from the pain and the anticipation thereof.

### RELATED APPLICATIONS

This application claims priority to U.S. Provisional Patent Application No. 61/656,702, filed Jun. 7, 2012, the contents of which are herein incorporated by reference in their entirety.

### BACKGROUND

Devices such as hypodermic needles, syringes, and other similar devices are generally designed in a manner that can create unnecessary pain, fear and anxiety. The pain is typically derived from the stimulus of the insertion of a sharp needle into the patient's skin, while the anxiety typically stems from the expectation of this pain. The pain and anxiety can lead to reactions such as jerks or tremors by the patient, which in turn can result in a suboptimal use of the device for the patient and technician using the device. Furthermore, the pain and anxiety resulting from this use can be traumatic for the patient leading to a phobic response. A way of diminishing the pain and/or anxiety related to hypodermic devices is therefore needed.

### SUMMARY

According to at least one exemplary embodiment, an injection device may be disclosed. The injection device may use a plurality of projections which may be placed against the skin to alleviate or distract from the anticipation, fear or pain of a shot. A vibration motor may be included within the device so that when desired the device and or the plurality of projections may vibrate. The vibration motor may be manual or electric and may be toggled or dialed across a gradient of vibration intensities. Decorative indicia may be provided on the exterior of the device. The exterior indicia may be removable and/or replaceable so as to enable a variety of designs. The injection device may further include noise making systems such as mp3 players, radios, bells, chimes, and rattles. The device may also include a chamber for syringes or needles which may allow the point of the needle to selectively extend or retract with respect to the plurality of projections.

According to at least one other exemplary embodiment, an injection device may be disclosed. The device can include a needle, which may be surrounded by a plurality of projections. The needle can further be adjustable between a first position and a second position. A vibration motor disposed within the device can impart vibration to the device. Decorative indicia may be provided on the exterior of the device. The device can facilitate reducing patient anxiety and pain by providing additional sensory stimuli to distract from the pain and the anticipation of an injection.

### BRIEF DESCRIPTION OF THE DRAWINGS

Advantages of embodiments of the present invention will be apparent from the following detailed description of the exemplary embodiments. The following detailed description should be considered in conjunction with the accompanying figures in which:

FIG. 1 is a side view of an exemplary embodiment of an injection device.

FIG. 2 is a front view of an exemplary embodiment of an injection device.

FIG. 3 shows a schematic of an exemplary embodiment of an injection device.

### DETAILED DESCRIPTION

Aspects of the present invention are disclosed in the following description and related figures directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

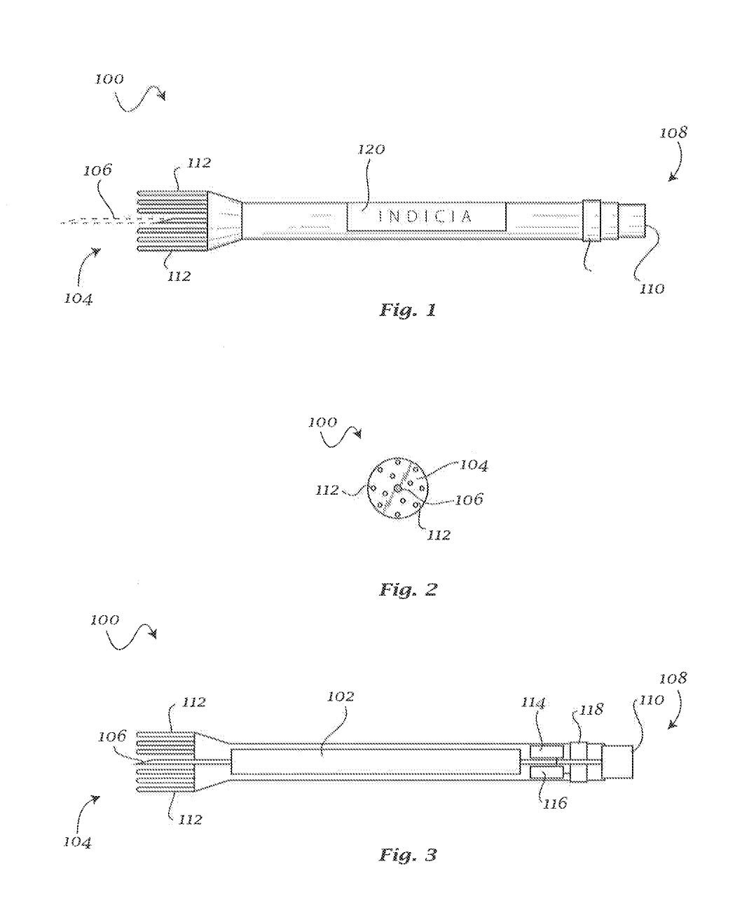

Generally referring to FIGS. 1-3, an injection device 100 may be disclosed. Device 100 may have any desired shape that allows it to function as described herein, for example a cylindrical shape. Additionally, device 100 may include a reservoir 102 disposed therein. Reservoir 102 may be hollow and may be adapted to contain a liquid, for example a medical solution. A first end 104 of device 100 may include at least one needle 106. A second end 108 of device 100 can include a user-operable mechanism 110 which may cooperate with reservoir 102 to facilitate administering the withdrawal or delivery of fluids to the patient via needle 106. For example, mechanism 110 may be a plunger, a push-button, or any other mechanism known in the art that enables device 100 to function as described herein. Other exemplary embodiments may have a specially designed cavity that may accommodate the use of any commercially available syringe or specific sizes of commercially available syringes.

Needle 106 can extend outwardly from first end 104, for example, substantially parallel to the longitudinal axis of device 100. Needle 106 can further extend substantially co-linearly with the longitudinal axis of device 100. First end 104 may further include a plurality of protrusions 112 extending outwardly therefrom. Protrusions 112 may have, for example, blunt or rounded distal ends but may have other ends. The length of protrusions 112 can be such that the protrusions contact the patient's skin while allowing needle 106 to extend under the patients skin so as to administer the desired hypodermic injection. The length of needle 106 may be adaptable for the particular type of hypodermic injection desired, e.g. a subcutaneous, intravenous, intramuscular, or other injection. Furthermore, for exemplary embodiments with a built in reservoir 102, the needle 106 may be removably coupled to device 100 so as to allow a variety of needles of different lengths to be used with device 100. Needle 106 may also be adapted to have a duller or sharper point than needles commonly used in the art, so as to reduce or minimize the pain stimulus.

In some exemplary embodiments, needle 106 may be movable between a first position wherein needle 106 is at least partially recessed within device 100, and a second position wherein needle 106 is at least partially extended away from device 100. In one exemplary embodiment, when needle 106 is in the first position, protrusions 112 may extend further than needle 106, which may allow protrusions 112 to contact the patient's skin. When needle 106 is moved to the second position, the needle 106 may extend beyond protrusions 112, thereby allowing the user to administer the injection. The position of needle 106 may be adjustable, for example, by mechanism 110. For example, when depressed, mechanism 110 may be adapted to extend needle 106 and deliver the fluid within reservoir 102 in a single action. In other exemplary embodiments, needle extensions, syringe extensions and fluid delivery may be provided by several actions of mechanism 110, or by separate mechanisms.

Device 100 may further be capable of vibration. The vibration may be generated by a vibrational motor 114. Motor 114 may be, for example, an eccentric mass motor or any other vibrational motor that allows device 100 to function as described herein. Power to the motor may be provided by a battery 116. The battery 116 may be removable and replaceable and may be disposed within the housing of device 100. Alternatively, battery 116 may be rechargeable, and device 100 may include structures adapted to charge battery 116, for example external contact points, induction coils, or a socket for a power adapter plug. In other exemplary embodiments, the vibration of device 100 may be manually facilitated or operated. For example, an eccentric mass disposed within the body of device 100 may be coupled to a user-operable member. Alternatively, any structure for manually vibrating the device 100 may be contemplated and provided as desired.

Motor 114 may be operable via a switch or dial 118 which may be provided on the exterior of device 100. In an exemplary embodiment, switch 118 may be provided as a ring rotatably and concentrically coupled to device 100, which the user may rotate to switch motor 114 on or off. In alternative embodiments, motor 114 may be toggled between an on or off position by any manner desired as would be understood by a person of ordinary skill in the art.

The device may further include indicia 120 which may aid in the relief of anxiety and fear that a patient could feel when experiencing or anticipating an injection. For example, for children, such indicia 120 can include, but are not limited to, visual representations of cartoon characters, combinations of color, cars, and sports or the like. In some exemplary embodiments, the device may also have the capability of producing audio as a means to alleviate a patient's anxiety and fear. In other exemplary embodiments, indicia 120 may be directed towards any age group or demographic, for example, adult or elderly patients.

Indicia 120 may further be detachably coupled to device 100, so as to allow the user to vary or customize the types of indicia 120. For example, a first indicia may be replaced with at least one second indicia depending on a patient's preference or reaction. The indicia 120 may be removably coupled via any desired fastening means known in the art, for example by clips, snaps, adhesive material, cling material and so forth.

In operation, a user may fill reservoir 102 with a desired liquid. In alternative embodiments, user may leave reservoir 102 empty, for example, if drawing fluids from a patient. User 102 may then attach any desired size needle 106 to device 100. Subsequently, the user may switch motor 114 on so as to impart vibration to device 100. The user may then contact the patient's skin with protrusions 112, and may activate mechanism 110 so as to extend the needle 106 beyond protrusions 112 and administer or draw the desired liquid.

The plurality of protrusions 112 can provide multiple contact points between device 100 and the patient's skin. This may provide additional sensory stimuli to the patient, thereby distracting the patient from the pain stimulus of the needle 106. The vibration provided by motor 114 may add yet more sensory stimuli, and further distract the patient from the pain stimulus. In the retracted position, needle 106 may not be as visible to the patient as in a typical hypodermic device, which may further lessen anxiety prior to injection. Indicia 120 may provide additional diversion for the patient, which may further lessen the patient's anxiety.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A injection device comprising:
an outer casing with a first end and a second end
a needle coupled to the first end of the outer casing;
a reservoir within the outer casing in fluid communication with the needle; and
a plunger proximate the second end of the outer casing, the plunger operable to expel or draw liquid into the reservoir through the needle.

2. The injection device as in claim 1, wherein a visible portion of the outer casing is capable of showing indicia.

3. The injection device as in claim 1, wherein the outer casing has a built in vibrator.

4. The injection device as in claim 1, wherein the outer casing has a built in sound generator.

5. The injection device as in claim 1, wherein the first end of the outer casing has a plurality of projections substantially parallel to the needle.

6. The injection device as in claim 1, wherein the needle may be detached from the outer casing and at least one second needle attached.

7. The injection device as in claim 2, wherein the visible portion of the outer casing is removable, and at least one second visible portion attached.

8. The injection device as in claim 5, wherein the needle may be moved between two or more stages comprising:
at least one first stage setting where the plurality of projections protrude farther from the outer casing than the needle;
at least one second stage setting where the needle protrudes farther from the outer casing than the plurality of projections; and
a mechanism controls a transition of the needle from the at least one first stage to the at least one second stage.

9. The injection device as in claim 8, further comprising:
a removable portion of the outer casing capable of showing indicia;
at least one removable second needle;
at least one sound generator; and
at least one vibrator.

10. A injection device comprising:
an outer casing;
a syringe housed within the outer casing; and
a plurality of projections on the outer casing arranged substantially parallel to the syringe.

11. The injection device as in claim 10, wherein the outer casing may be opened to receive the syringe and closed to secure the syringe within the outer casing.

12. The injection device as in claim 10, wherein a visible portion of the outer casing is capable of showing indicia.

13. The injection device as in claim 10, wherein the outer casing has a built in vibrator.

14. The injection device as in claim 10, wherein the outer casing has built in sound generator.

15. The injection device as in claim 12, wherein the visible portion of the outer casing is removable, and at least one second visible portion attached.

16. The injection device as in claim 10, wherein the syringe may be moved between two or more stages comprising:
at least one first stage setting where the plurality of projections protrude farther from the outer casing than the syringe;
at least one second stage setting where the syringe protrudes farther from the outer casing than the plurality of projections; and
wherein a mechanism controls a transition of the syringe from the at least one first stage to the at least one second stage.

17. The injection device as in claim 16, further comprising:
a removable portion of the outer casing capable of showing indicia;
at least one sound generator;
at least one vibrator
wherein the outer casing may be opened to receive the syringe and closed to secure the syringe within the outer casing;

18. The injection device as in claim 10, wherein the syringe may be removed and replaced by at least one second syringe without opening or closing the outer casing.

19. A injection device comprising:
a syringe;
and an outer casing at least partially encompassing the syringe, wherein the outer casing further includes at least one of visual, audio, and physical stimuli.

20. The injection device of claim 19, further comprising:
a plurality of projections on the outer casing arranged substantially parallel to the syringe; and
a mechanism controlling a position of the syringe with respect to the plurality of projections.
